(19) United States
(12) Patent Application Publication
(10) Pub. No.: US 2003/0195598 A1
(43) Pub. Date: Oct. 16, 2003

(54) Disposable compress

(76) Inventors: Sabeto A. DiRoma, Oakdale, CT (US); Frank P. Recchia, New Haven, CT (US)

Correspondence Address: Law Offices of Raymond A. Nuzzo, LLC, 579 Thompson Avenue, East Haven, CT, 06512, US

(21) Appl. No.: 10/120,717

(22) Filed: Apr. 11, 2002

(51) Int. Cl. A61F 7/00

(52) U.S. Cl. 607/114

(57) ABSTRACT

A disposable cold pack comprising a durable flexible container having an interior region, a breakable envelope contained within the interior region, a predetermined amount of liquid contained within the envelope wherein the liquid consists of water, and solid materials contained within the interior region. The solid materials consist of a predetermined amount of urea and a predetermined amount of potassium chloride. When the envelope is broken so as to release water into the interior region, the water dissolves a substantial amount of the solid materials thereby producing an endothermic reaction which cools the water.

### BACKGROUND OF THE INVENTION

1. Field of the Invention

The present invention generally relates to a disposable instant cold compress, and more particularly to a disposable cold compress that is capable of initially producing cold by thermo-chemical reaction.

2. Description of Related Art

Cold compresses or cold packs have long been used to apply cold to human or animal body parts. Such compresses and cold packs are described in U.S. Pat. Nos. 3,887,346, 3,950,158, 4,081,256, 5,391,198, 5,534,020, 5,545,197, 6,099,555 and 6,233,945.

Many prior art cold compresses and cold packs utilize ammonium nitrate. There are several significant disadvantages in using ammonium nitrate. One disadvantage is that ammonium nitrate is listed as a dangerous and/or hazardous substances on the Material Safety Data Sheet (MSDS). Thus, handling, transporting and storing ammonium nitrate is both time consuming and expensive. Furthermore, IATA has included ammonium nitrate in its Dangerous Goods Regulations. Thus, many air carriers and commercial airlines will not transport ammonium nitrate. Additionally, the Federal Bureau of Investigation, in its Congressional Statement on the Threat of Terrorism to the United States, dated May 10, 2001, has identified ammonium nitrate as a chemical used by right-wing extremist groups and terrorists in making explosive devices.

Another potential problem with the use of ammonium nitrate, is that in some situations, the ammonium nitrate produces such extremes of cold temperatures that the user may experiences frost bite.

What is needed is a disposable cold pack that does not utilize ammonium nitrate, but which provides the sufficient cold to achieve the desired effect on the human or animal body part to which the cold compress is applied. Such a disposable cold compress should utilize a minimum number of different chemicals and should be economical to manufacture.

### SUMMARY OF THE INVENTION

The present invention is directed to, in one aspect, a disposable cold pack, comprising a durable flexible container having an interior region, a breakable envelope contained within the interior region, a predetermined amount of liquid contained within the envelope wherein the liquid consists of water, and solid materials contained within the interior region. The solid materials consist of a predetermined amount of urea and a predetermined amount of potassium chloride. When the envelope is broken so as to release water into the interior region, the water dissolves a substantial amount of the solid materials thereby producing an endothermic reaction which cools the water.

In a related aspect, the present invention is directed to a disposable cold pack, comprising a durable flexible container having a first compartment, a second compartment adjacent the first compartment, and a breakable seal separating the first and second compartments. A predetermined amount of liquid is contained within the first compartment. The liquid consists of water. Solid materials are contained within the second compartment. The solid materials consist of a predetermined amount of urea and a predetermined amount of potassium chloride. When the breakable seal is broken, the water enters the second compartment and dissolves a substantial amount of the solid materials thereby producing an endothermic reaction which cools the water.

### BRIEF DESCRIPTION OF THE DRAWINGS

The figures are for illustration purposes only and are not drawn to scale. The invention itself, however, both as to organization and method of operation, may best be understood by reference to the detailed description which follows taken in conjunction with the accompanying drawings in which:

FIG. 1 is a perspective view of the compress according to one embodiment of the present invention;

FIG. 2 is a front view of the compress shown in FIG. 1;

FIG. 3 is a view taken along line 3-3 in FIG. 2.

FIG. 4 is a view of the compress shown in FIG. 3 after activation.

FIG. 5 is a chart that illustrates time-versus-temperature data of the compress of the present invention for various concentrations of potassium chloride.

FIG. 6 is a chart that illustrates time-versus-temperature data of the compress of the present invention for various particle sizes of potassium chloride.

FIG. 7 is a front view of a compress in accordance with another embodiment of the invention.

### DETAILED DESCRIPTION OF THE INVENTION

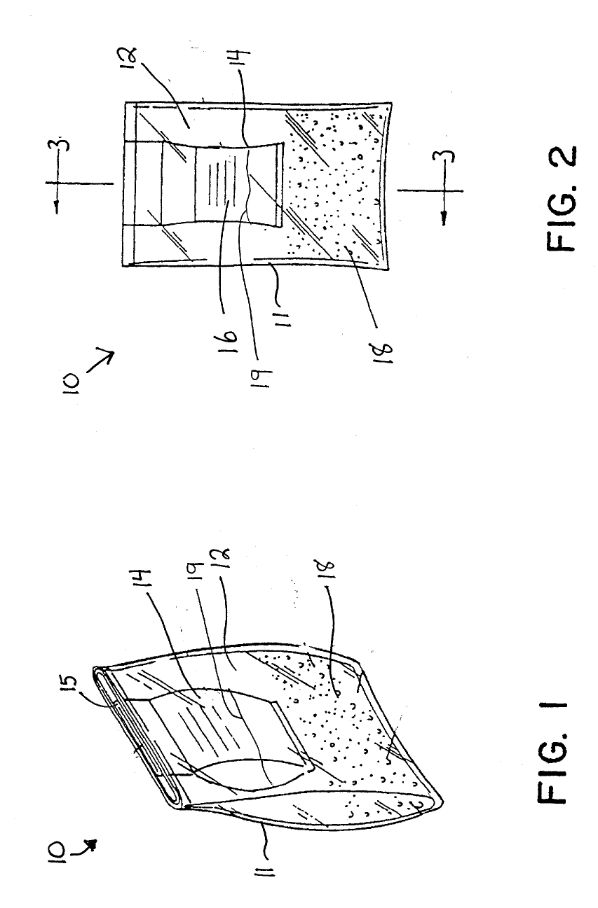

Referring to FIGS. 1-3, there is shown one embodiment of the disposable compress or cold pack of the present invention. Disposable cold pack 10 comprises an outer, durable, flexible, bag-like, plastic container 11 having an interior 12, and an inner plastic envelope 14 that is disposed within interior 12. Such a container is described in U.S. Pat. No. 5,534,020. Envelope 14 is attached to seam 15 of container 12. In one embodiment, plastic envelope 14 is a saran-coated polymylar envelope. Plastic envelope 14 is scored on its exterior surface 16 so that it will break when a pressure of about 25 pounds per square inch is applied to it. Interior 12 contains solid materials 18 therein. Solid materials 18 consist of a predetermined amount of urea and potassium chloride. Plastic envelope 14 contains only a predetermined amount of liquid 19. Liquid 19 consists of water.

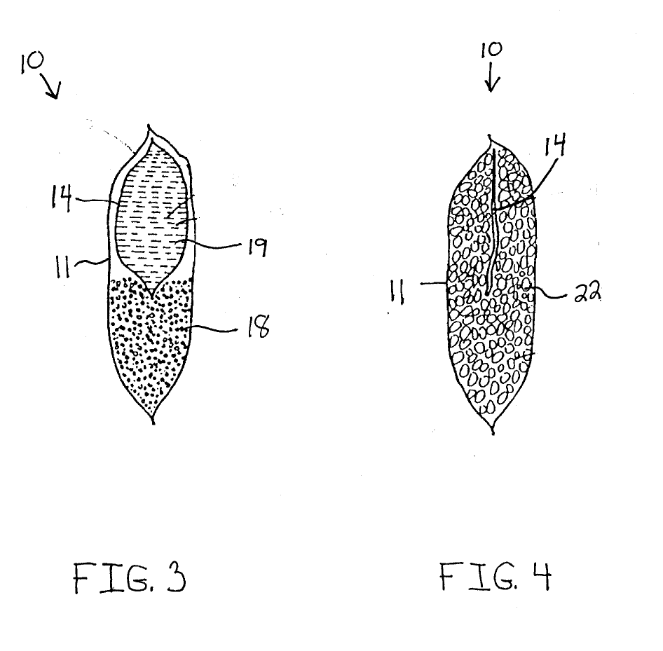

Referring to FIGS. 3 and 4, envelope 14 is relatively small, made out of a material durable enough not to break or rupture prematurely but weak enough to break or rupture when direct pressure is applied. Referring to FIG. 4, when envelope 14 is ruptured and water is released into interior 12, the potassium chloride and urea mix with water 19 so as to produce mixture 22. As a result, a substantial amount of urea and potassium chloride is dissolved in the water thereby producing an endothermic reaction. Shaking cold pack 10 facilitates mixture of the urea and potassium chloride with the water. As a result, the water cools. Cold pack 10 can then be applied to a human or animal body part. Due to the flexibility of cold pack 10, the cold pack is easy to wrap on the body part.

In a preferred embodiment, the predetermined amount of water 19 is between about 4.25 ounces (120 grams) and 4.50 ounces (128 grams), inclusive. In one embodiment, the predetermined amount of water is about 4.42 ounces (or 125 grams).

In a preferred embodiment, the predetermined amount of urea is between 4.0 ounces (113 grams) and 5.0 ounces (142 grams), inclusive. In one embodiment, the predetermined amount of urea is about 4.24 ounces (or 120 grams). In one embodiment, the urea is in the form of urea prills.

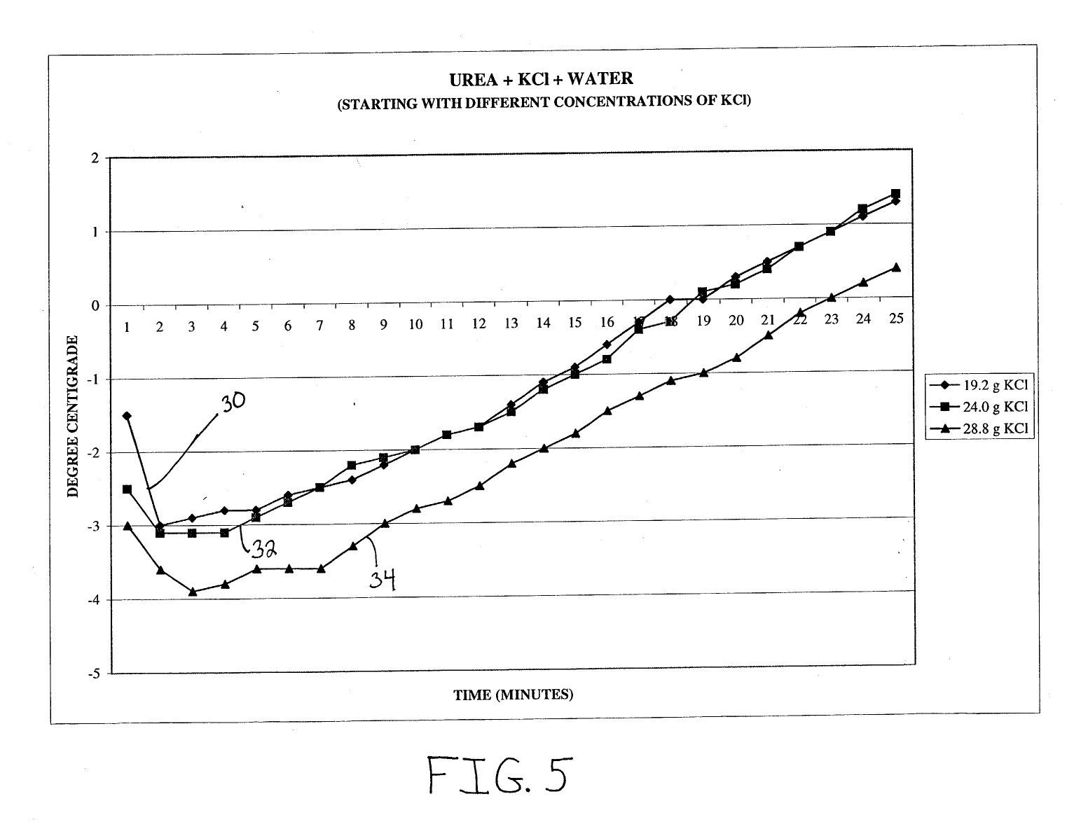

In a preferred embodiment, the predetermined amount of potassium chloride is between 19.0 grams (0.67 ounces) and 30.0 grams (1.06 ounces), inclusive. In one embodiment, the predetermined amount of potassium chloride is 19.2 grams (0.68 ounces). In another embodiment, the predetermined amount of potassium chloride is 24.0 grams (0.85 ounces). In a further embodiment, the predetermined amount of potassium chloride is 28.8 grams. Referring to FIG. 5, there is shown temperature-versus-time data for about 4.24 ounces (120 grams) of urea prills, 4.42 ounces (125 grams) of water, and various concentrations of potassium chloride. Curve 30 indicates temperature-versus-time data for 19.2 grams (0.68 ounces) of potassium chloride. Curve 32 indicates temperature-versus-time data for 24.0 grams (0.85 ounces) of potassium chloride. Curve 34 indicates temperature-versus-time data for 28.8 grams (1.02 ounces) of potassium chloride.

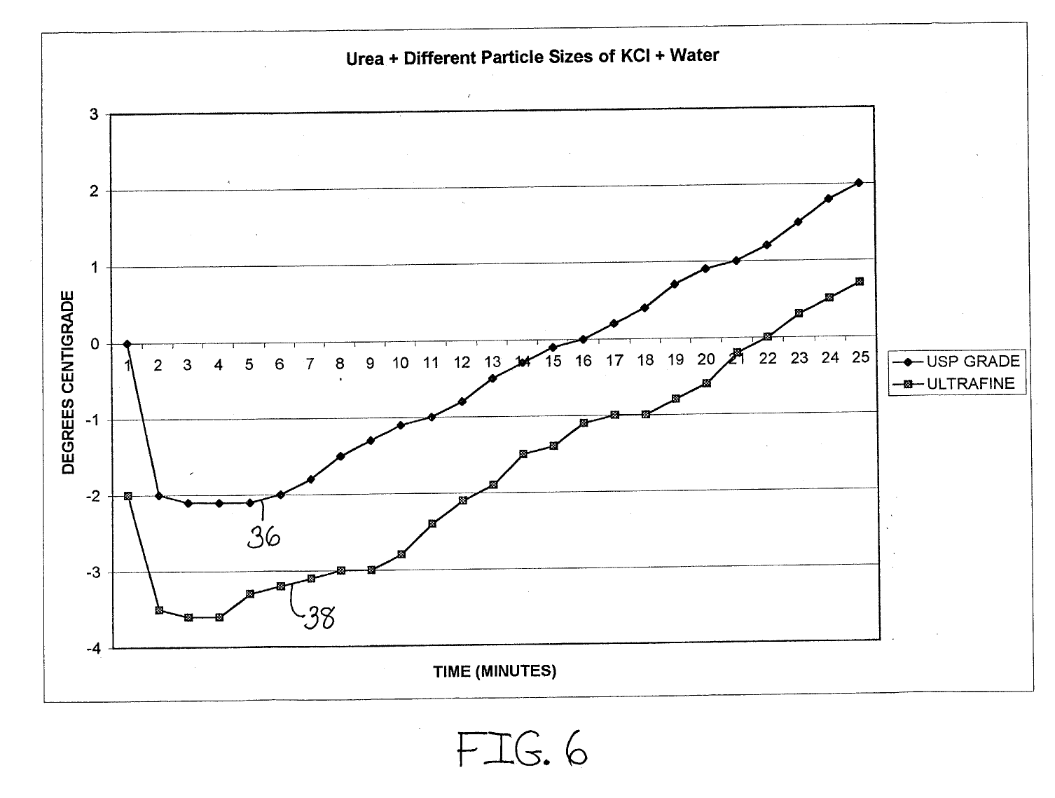

It has been found that as the size of the particles of potassium chloride decrease, the temperature also decreases. This characteristic is illustrated in FIG. 6 which shows temperature-versus-time data for USP grade potassium chloride, indicated by curve 36, and for ultra fine potassium chloride, indicated by curve 38. The data represented by curve 36 is based on the following parameters: a urea prill concentration of about 4.24 ounces (120 grams), 4.42 ounces (125 grams) of water, and 0.85 ounces (24 grams) of USP grade potassium chloride. The data represented by curve 38 is based on the following parameters: a urea prill concentration of about 4.24 ounces (120 grams), 4.42 ounces (125 grams) of water, and 0.85 ounces (24 grams) of ultra fine potassium chloride.

In a preferred embodiment, cold pack 10 of the present invention utilizes 4.24 ounces (120 grams) of urea prills, 28.8 grams (1.02 ounces) of ultra fine potassium chloride, and 4.42 ounces (125 grams) of water.

In an alternate embodiment, envelope 14 is not attached to seam 15 of container 11. Instead, envelope 14 is disposed loosely in interior 12.

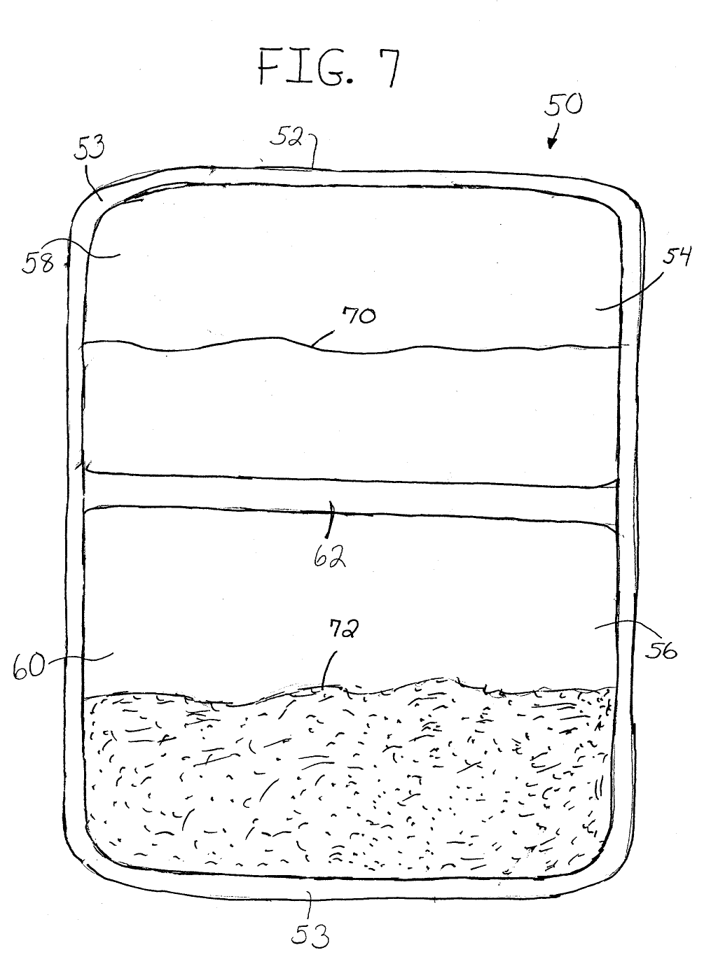

Referring to FIG. 7, there is shown an alternate embodiment of the compress of the present invention. Cold pack 50 generally comprises durable, flexible, bag-like, plastic container 52 having an outer seal 53 and adjacent compartments 54 and 56. Compartment 52 has an interior region 58. Similarly, compartment 56 has an interior region 60. Interior regions 58 and 60 are separated by seal 62. Seal 62 is fabricated from a material durable enough not to break prematurely but weak enough to break when direct pressure is applied thereto. Interior region 58 contains only a predetermined amount of liquid 70 which consist of water 70. Interior region 60 contains only solid materials 72 that consist of a predetermined amount of urea and potassium chloride. The amounts of water, urea and potassium chloride described in the foregoing description pertaining to cold pack 10 may be used with cold pack 50.

In order to rupture or break seal 62, the user squeezes section 58 so the water therein is forced against and ruptures seal 62. When seal 62 is ruptured, water from interior region 58 is released into interior region 60. As a result, a substantial amount of the potassium chloride and urea are dissolved in the water thereby producing an endothermic reaction. Shaking cold pack 50 facilitates mixture of the urea and potassium chloride with the water. The endothermic reaction causes the water to cool. The cold pack can then be applied to a human or animal body part. Due to the flexibility of cold pack 50, the cold pack is easy to wrap on the body part.

Thus, the cold pack of the present invention:

a) provides cold temperatures sufficient to achieve the medical purpose for which the cold pack is being used;

b) does not utilize ammonium nitrate thereby eliminating the problems, issues and disadvantages associated with ammonium nitrate; and

c) can be economically manufactured and provided to consumers at a reasonable cost.

While the present invention has been particularly described, in conjunction with a specific preferred embodiment, it is evident that many alternatives, modifications and variations will be apparent to those skilled in the art in light of the foregoing description. It is therefore contemplated that the appended claims will embrace any such alternatives, modifications and variations as falling within the true scope and spirit of the present invention.

Thus, having described the invention, what is claimed is:

1. A disposable cold pack, comprising:
a durable flexible container having an interior region;
a breakable envelope contained within the interior region;
a predetermined amount of liquid contained within the envelope, the liquid consisting of water; and
solid materials contained within said interior region, said solid materials consisting of a predetermined amount of urea and a predetermined amount of potassium chloride;
wherein when said interior envelope is broken so as to release water into said interior region, said water dissolves a substantial amount of said solid materials thereby producing an endothermic reaction which cools the water.

2. The disposable cold pack according to claim 1 wherein the predetermined amount of water is between about 4.25 and 4.50 ounces.

3. The disposable cold pack according to claim 1 wherein the predetermined amount of urea is between about 4.0 and 5.0 ounces.

4. The disposable cold pack according to claim 4 wherein the predetermined amount of urea is about 4.42 ounces.

5. The disposable cold pack according to claim 1 wherein the predetermined amount of potassium chloride is between about 19.0 and 30.0 grams.

6. The disposable cold pack according to claim 1 wherein the predetermined amount of potassium chloride is about 28.8 grams.

7. The disposable cold pack according to claim 1 wherein the urea is in the form of urea prills.

8. The disposable cold pack according to claim 1 wherein the potassium chloride is in ultra-fine powder form.

9. The disposable cold pack according to claim 1 wherein:
said potassium chloride is in ultra-fine powder form;
said urea is in the form of urea prills;
said predetermined amount of liquid is about 4.42 ounces;
said predetermined amount of urea is about 4.24 ounces; and
said predetermined amount of potassium chloride is about 28.8 grams.

10. A disposable cold pack, comprising:
a durable flexible container having a first compartment,
a second compartment adjacent the first compartment, and a breakable seal separating the said first and second compartments;
a predetermined amount of liquid contained within said first compartment, said liquid consisting of water; and;
solid materials contained within said second compartment, said solid materials consisting of a predetermined amount of urea and a predetermined amount of potassium chloride;
wherein when said breakable seal is broken, said water enters the second compartment and dissolves a substantial amount of said solid materials thereby producing an endothermic reaction which cools the water.

11. The disposable cold pack according to claim 10 wherein the predetermined amount of water is between about 4.25 and 4.50 ounces.

12. The disposable cold pack according to claim 10 wherein the predetermined amount of urea is between about 4.0 and 5.0 ounces.

13. The disposable cold pack according to claim 12 wherein the predetermined amount of urea is about 4.42 ounces.

14. The disposable cold pack according to claim 10 wherein the predetermined amount of potassium chloride is between about 19.0 and 30.0 grams.

15. The disposable cold pack according to claim 14 wherein the predetermined amount of potassium chloride is about 28.8 grams.

16. The disposable cold pack according to claim 10 wherein the urea is in the form of urea prills.

17. The disposable cold pack according to claim 10 wherein the potassium chloride is in ultra-fine powder form.

18. The disposable cold pack according to claim 10 wherein:
said potassium chloride is in ultra-fine powder form;
said urea is in the form of urea prills;
said predetermined amount of liquid is about 4.42 ounces;
said predetermined amount of urea is about 4.24 ounces; and
said predetermined amount of potassium chloride is about 28.8 grams.
